(19) United States
(12) Patent Application Publication
(10) Pub. No.: US 2011/0044959 A1
(43) Pub. Date: Feb. 24, 2011

(54) NOVEL COMPOSITION OF STEM CELLS FOR TRANSPLANTATION TOLERANCE

(76) Inventors: H.L. Trivedi, Ahmedabad (IN); Aruna Vanikar, Ahmedabad (IN)

Correspondence Address: Davidson, Davidson & Kappel, LLC, 485 7th Avenue, 14th Floor, New York, NY, 10018, US

(21) Appl. No.: 12/922,636

(22) PCT Filed: Mar. 13, 2009
(86) PCT No.: PCT/IN09/00175

§ 371 Date: Sep. 14, 2010

(30) Foreign Application Priority Data
Mar. 15, 2008 (IN) 1507/MUM/2008

(51) Int. Cl. A61K 35/14; C12Q 1/06; A61P 37/06

(52) U.S. Cl. 424/93.7; 435/39

(57) ABSTRACT

The present invention provides a simple, economical yet efficient method of creating transplant tolerance in organ transplant patients without the continuous need for costly immunosuppressive drugs with serious adverse effects. The invention essentially deals with the administration of a novel composition to the patient which consists of adipose tissue derived Mesenchymal Stem Cells (MSC) combined with bone marrow derived Haematopoietic Stem Cells(HSC) and MSC and peripheral blood stem cells (PBSC). This helps in creating transplant tolerance ie. Stable adequate allograft function with minimum/no rejection using very low dose of immunosuppressive medication. The invention also deals with a simple method of isolating Mesenchymal Stem cells from human adipose tissue without using any xenogenic material.

### FIELD OF INVENTION

This invention essentially deals with a novel method of obtaining mesenchymal stem cells (MSC) from adipose tissue and their use in combination with bone marrow and peripheral blood derived hematopoietic and mesenchymal stem cells for creating “transplantation tolerance” which means transplantation using minimum/no immunosuppressive medication.

### BACKGROUND

Transplantation has become an acceptable therapeutic modality for patients dying of organ failure. Unfortunately, all these patients have to be maintained on life long immunosuppressive medications. This entire exercise of finding an organ donor, finances incurred thereof and the post-operative management is emotionally exhausting and financially prohibitive for an average Indian family. Even if they manage to cross all these hurdles, complications in the form of infections and/or malignancy take away the grafted organ as well as life of the patients many a times. The only logical answer to these problems is transplantation with minimum/no immunosuppressive medications (Tolerance).

We therefore embarked on our research on “tolerance” seriously since August 1998. The principal theme of our research is to perform donor stem cell transplantation in the recipient before organ transplantation. Successful stem cell transplantation will protect the grafted organ from being rejected without any immunosuppressive drugs. We kept on modifying our tolerance research protocol till a safe and effective protocol emerged.

Since last 50 years numerous medical scientists including Sir Peter Medawar (Nobel laureate) have been working on the theme of “tolerance” in different animal models. None of these themes could successfully be transferred to human model because the human immunobiology is much more complex than that of animals and therefore is difficult to implement the same work in humans. We are happy to state here that we, the Ahmedabad team at IKDRC-ITS, have more than 1000 patients transplanted across MHC barriers (which means irrespective of genetic
compatibility and immunologic matching with donor) on minimum immunosuppression (called prope tolerance). Most of them have steady and adequate graft function without any problem and some of them are living normal life without any immunosuppressive medication. The major advantage of this research is that it has given the gift of life to patients dying of organ failure and that too at nominal cost! However we were not able to achieve reproducible results. The problem was unavailability of adequate number of MSC which improve hematopoietic stem cell (HSC) grafting and thereby creating better tolerance. We found out that adipose tissue was an easy source of procuring adequate number of MSC. Hence we modified our research and developed our own technique of isolating MSC from adipose tissue of organ donor.

### OBJECTIVES AND SUMMARY OF THE INVENTION

The primary objective of the present invention is to find out an alternative and adequate source of Donors only mesenchymal stem cells (MSCs) which can be routinely used for creating transplantation “tolerance”. Another objective of the present invention is to develop a practical and convenient method of isolating and differentiating the MSCs from the selected source. Yet another objective of the present invention is to provide a composition of different stem cells and a method of administration to the patient to achieve most desirable and consistent results.

The present invention describes for the first time—A novel composition of adipose tissue derived MSC, bone marrow derived HSC and MSC and peripheral blood stem cells (PBSC) which help in creating transplantation tolerance (stable adequate allograft function with minimum/no rejection using very low doze of immunosuppressive medication).
These cells are transplanted in portal circulation using our own technique of omental vein canulation via mini-laparatomy. These cells are transplanted under non-myeloablative minimal conditioning using donor specific leucocyte transfusions, anti-T and anti-B cell antibodies to the recipient and target specific irradiation of 1000 CGY to sub-diaphragmatic lymph nodes, part of pelvic and hip bones and thoraco-lumbar vertebrae of the recipient before transplanting stem cells.

This is the first time that mesenchymal stem cells MSCs have been derived in vitro from human adipose tissue without using any xenogenic material. There are reports of generating MSC from embryonic stem cells or cord blood or bone marrow (BM) using xenogenic material. However MSC have not been generated without the use of xenogenic material

### DETAILED DESCRIPTION OF THE PRESENT INVENTION

We observed during our continued research that MSC improve HSC grafting and that adipose tissue is a good and easily accessible and available source of MSC. MSC are not available in large number from any source other than adipose tissue.

We developed a novel technique for isolating and culturing MSC from the adipose tissue of the organ donor. We have successfully used a unique combination of stem cells i.e. adipose tissue derived MSC, bone marrow derived HSC and MSC and peripheral blood stem cells (PBSC) for creating transplantation tolerance. Tolerance is associated with grafting of about 10% HSC in bone marrow. PBSC are a rich source of T-lymphocytes, which are also essential for stem cell grafting. MSC act as big brother of HSC. They work as scaffoldings and help in inter-organ chemotactic transportation of HSC.

According to Mesenchymal and Tissue Stem Cell Committee of the International Society for Cellular Therapy, MSC have to be plastic adherent when maintained under standard culture condition, they must exhibit adipogenic, chondrogenic and osteogenic differentiation potential, they must express CD90, CD73 and CD105 markers positive and same way also they must lack expression of markers for hematopoietic lineages of cells which include CD45, CD34, CD 14, CD11b, CD29, HLA-DR, c-kit. Our cell lines fulfill these criteria.

The effect of adipose tissue derived MSC (hAD-MSC) is dose dependent, 1:20 ratio of hAD-MSC: HSC is required for engraftment of the cells, more the ratio, better the engraftment will be. Our results confirm these lab findings.

Attempts to induce transplantation tolerance by different strategies using drugs and hematopoietic stem cells have been tried unsuccessfully. This is the first successful attempt of achieving transplantation tolerance by using unique composition of stem cells.

These cells are transplanted in portal circulation using our own technique of omental vein canulation via mini-laparatomy.

These cells are transplanted under non-myeloablative minimal conditioning using Donor specific leucocyte transfusions, anti-T and anti-B cell antibodies to the recipient and target specific irradiation of 1000 CGY to sub-diaphragmatic lymph nodes, part of pelvic and hip bones and thoracic thoraco-lumbar vertebrae of the recipient before transplanting stem cells.

Thus, this unique composition of adipose tissue derived MSC, bone marrow derived HSC and MSC and peripheral blood stem cells PBSC is essential to create transplantation tolerance associated with grafting, under above mentioned conditioning (of anti T/B cell antibodies and target specific irradiation).

Following technique was developed and used for collecting and culturing MSC cells from adipose tissue.

### TECHNIQUE:

### Isolation of MSC After Collection From Adipose Tissue:

After their informed consent was obtained, 1.5 gram of adipose tissue was resected from anterior abdominal wall of donors under local anesthesia after making a small incision on left lateral side below umbilicus. Sutures were taken after hemostasis was secured.

This adipose tissue was collected in the following medium:
1. 20 ml α-MEM
2. 5 ml, 20% human albumin
3. 20 μl, penicillin (200000 units/ml)
4. 20 μl, streptomycin (200000 units/ml)
5. 10 μl, Cefotaxime (1 gm/ 5 ml)
6. 10 μl, Fluconazole, 100 mg/dl.

The adipose tissue was minced with knife into tiny pieces. Then it was transferred in to the above medium with addition of collagenase type I, 10 mg per every 10 ml. It was then incubated at 37° C. for 1 hr. on shaker with 35 RPM for digestion. The entire contents of the medium processed in Petri dish were transferred to 15 ml centrifuge tubes, centrifuged at 780 RPM for 8 minutes. The supernatant and pellets were separately cultured in the above medium on 100 sq. cm and 25 sq. cm. cell+culture dishes (Sarsted, USA) respectively, at 37° C. with 5% CO2 for 8 days. The cells were subjected to 3 passages (medium changed on alternate days) and at the end of 3^{rd} passage, they were harvested by means of trypsinization (0.25% trypsin EDTA solution, made up of 0.25% trypsin and 0.2% sodium EDTA powder, HiMedia, India) after washing with 1 N phosphate buffered saline (PBS).

Collected cells were checked for viability, sterility and cell counts and flow cytometric analysis. CD 45(Per CP) negative and CD90 (PE) positive tests were carried out. These cells were mixed with cultured bone marrow and peripheral blood stem cells PBSC and total contents were infused in portal circulation.

### RESULTS OF PRESENT INVENTION

### Details of the Patients are Given Below:

Total Number of kidney Transplanted Patients Using MSC+BMSC+PBSC: 60
Age: 33.5 (range: 8-59) yrs
Gender: M: F: 49:11
Donor age: 46.4 (range: 20-67) yrs
HLA MATCH: 0/6: 2, 1/6: 11, 2/6: 10, 3/6: 28, 4/6: 5, 5/6: 2, 6/6: 2

### Information About the Stem Cells Infused:

MEAN BM CD34+: 0.23 (range: 0.01-4.01) (STDEV-0.37)
MEAN CD 45−/90+:
BM: 0.42 (RANGE: 0.02-0.84) (STD. DEV- 0.23)
BM+MSC: 21.67 (range: 3.06-63.73) (STDEV MSC-18.3). (These are not present in PBSC)
Mean MSC+BM CD 45−/73+ (Not found in BM alone):
6.39 (range: 0.1-23.82) (STD DEV: 6.37)
FOLLOW UP:
DAYS: 99.86 (19-184)
S.Creatinine (mg %):1.27(0.63-2.3) (S.D.: 0.32)

### Chimerism:

Tested by Fluorescent in situ hybridization (FISH): 7.2% (range: 5-14.7%)
CD3 dim (natural suppressor cells): 2.92% (range: 1.47-5.14%) (S.D.: 1.33)
CD 19+: 0.33 (range: 0.01-1.53%) (S.D.: 0.47)
CD 25+: 0.49 (range: 0.21-1.06%) (S.D.: 0.26)

1. A composition for inducing Transplantation Tolerance in patients undergoing transplantation, the composition comprising human adipose tissue derived Mesenchymal Stem Cells (MSC), bone marrow derived Mesenchymal Stem Cells (MSC) and Hematopoietic Stem Cells (HSC) and Peripheral Blood Stem Cells (PBSC) obtained from a kidney donor.

2. The composition of claim 1, wherein a ratio of the human adipose derived Mesenchymal Stem Cells to the Haematopoietic Stem Cells (HSC) is 1:20.

3. The composition of claim 1 for parenteral administration to patients undergoing Kidney transplants.

4. The composition of claim 3 for administration to patients, through portal circulation by infusion.

5. The method for creating ‘Transplant Tolerance’ in patients undergoing transplantation by administration by infusion of a composition comprising Human Adipose tissue derived Mesenchymal Stem Cells (MSC), bone marrow derived Mesenchymal Stem Cells (MSC), Haematopoietic Stem Cells (HSC) and Peripheral Blood Stem Cells (PBSC).

6. The method of claim 5, wherein the ratio of Human Adipose derived Mesenchymal Stem Cells to Haematopoietic Stem Cells (HSC) is 1:20.

7. The method of claim 5, wherein the administration is by infusion into portal circulation.

8. The method of claim 5, wherein the transplantation is kidney transplantation.

9. A process of obtaining Human Adipose tissue derived Mesenchymal Stem Cells (MSC) comprising steps of:
(a) collecting of donor adipose tissue in a medium free from a Xenogenic material and comprising α-MEM, Human albumin, sodium pyruvate, an antibiotics and at least one antifungal;
(b) mincing of adipose tissue into pieces and transferring the same to the medium free from the Xenogenic material along with collagenase type 1;
(c) incubating and digesting a mixture of step (b) for approximately 1 hour on a shaker;
(d) transferring the the mixture of step (b) to centrifuge tubes after the incubating and digesting, and centrifuging the mixture after the transfer to obtain a supernatant and pellets;
(e) culturing the supernatant and the pellets separately in the medium free from the Xenogenic material on culture dishes at 37 degree centigrade with 5% CO2 preferably for 8 days changing the medium free from the Xenogenic material on alternate days;
(f) harvesting cells formed in step (e) by means of trypsinization after washing with suitable buffer;
(g) collecting the harvested cells and checking viability, sterility and counts and carrying out flow cytometric analysis for CD45 negative/90 positive/73 positive events.

10. The process of claim 9, wherein the cells cultured in step (e) may be subjected to 3 passages during the culturing.

11. The composition of claim 2, for parenteral administration to patients undergoing Kidney transplants.

12. The composition of claim 11, for administration to patients, through portal circulation by infusion.

13. The method of claim 6, wherein the administration is by infusion into portal circulation.

14. The method of claim 6, wherein the transplantation is kidney transplantation.

15. The method of claim 7, wherein the transplantation is kidney transplantation.

16. The method of claim 5, wherein the transplantation is a solid organ transplantation.

17. The method of claim 6, wherein the transplantation is a solid organ transplantation.

18. The method of claim 7, wherein the transplantation is a solid organ transplantation
